(19) United States
(12) Patent Application Publication
(10) Pub. No.: US 2015/0064206 A1
(43) Pub. Date: Mar. 5, 2015

(54) COMPOSITIONS FOR TREATING UVEITIS

(71) Applicants: Arnaud FOUSSAT, Valbonne (FR); Nathalie BELMONTE, Nice (FR); Helene ASNAGLI, Nice (FR); Julie GERTNER - DARDENNE, Grasse (FR); Marie JACQUIN, Vence (FR); Marie-Francoise HUBERT, Jozerand (FR)

(72) Inventors: Arnaud FOUSSAT, Valbonne (FR); Nathalie BELMONTE, Nice (FR); Helene ASNAGLI, Nice (FR); Julie GERTNER - DARDENNE, Grasse (FR); Marie JACQUIN, Vence (FR); Marie-Francoise HUBERT, Jozerand (FR)

(73) Assignee: TXCELL, Valbonne (FR)

(21) Appl. No.: 14/524,406

(22) Filed: Oct. 27, 2014

Related U.S. Application Data
(63) Continuation-in-part of application No. 12/989,857, filed on Oct. 27, 2010.
(60) Provisional application No. 61/048,309, filed on Apr. 28, 2008.

(30) Foreign Application Priority Data
Apr. 28, 2008 (EP) 08305133.4

(51) Int. Cl. A61K 35/14; A61K 39/00; A61K 35/28

(52) U.S. Cl. CPC A61K 35/17; A61K 35/28; A61K 39/0008; A61K 2035/122
USPC 424/184.1

(57) ABSTRACT

Compositions including human Treg cells directed to an eye-associated antigen and methods for treating uveitis.

### FIELD OF THE INVENTION

The present invention relates to the field of treatment of uveitis. The present invention relates in particular to methods for treating uveitis, using a medicament comprising human regulatory T cells, preferably Tr1 cells directed against an eye-associated antigen.

### BACKGROUND

Uveitis is an inflammatory condition of the eye and the fourth most common cause of blindness among the working-age population in the developed world, behind congenital, degenerative and diabetes with accidents in fifth place. Visual loss in uveitis patients occurs during the acute inflammation phase, which is a time when the inflammation associated with the disease flares up. This is what has the most deleterious effect on the eye. The comorbidities of uveitis include cataracts, glaucoma, retinal vascular ischemia and in particular macular oedema, which can result in irreversible vision loss.

Uveitis affects mainly the uvea, or middle layer of the eye but the disease can also affect the lens, retina, optic nerve, and vitreous chamber, producing reduced vision or blindness, and can involve the full eye (panuveitis) or a segment of the eye (anterior, intermediate or posterior). Uveitis can be caused by systemic autoimmune disorders, such as Behcet's disease and Vogt-Koyanagi-Harada (VKH) disease; infections, including toxoplasmosis and syphilis; or trauma and surgery. However, the majority of uveitis cases are idiopathic, meaning that the cause is unknown. It can happen at all ages and primarily affects people between 17-60 years old. Infectious Uveitis (IU) corresponds to the majority of cases in developing countries while Non-Infectious Uveitis (NIU) represents the majority of cases in developed countries. Indeed, NIU accounts for 65 to 85% of uveitis cases.

Uveitis can be characterized according to the anatomical location of inflammation (anterior, intermediate, posterior or panuveitis) as well as the course of the disease. Acute uveitis presents itself by an episode that is of sudden onset and limited duration. Repeated episodes of uveitis that are separated by inactive periods but do not require treatment for three or more months are classified as recurring uveitis. Chronic uveitis is a form of persistent uveitis in which relapse occurs less than three months after the discontinuation of treatment. Both infectious and non-infectious aetiologies can develop into acute and chronic uveitis.

Depending on the exact location of the inflammation, potential underlying conditions, and factors such as the severity and course of the disease, the treatment of uveitis patients needs to be adapted to the individual. Currently, all approved treatments remain steroid based even though immunosuppressors and biologics are also used “off-label” to try to manage NIU.

While steroid therapy normally provides fast initial relief of symptoms in uveitis, the effect is limited, insufficient for the more severe cases and associated with significant local and systemic side effects including increased intra-ocular pressure, weight gain, hypertension, diabetes, and delayed wound healing, that more targeted treatments could avoid. Moreover, steroid treatment is clearly unsatisfactory to adequately manage NIU with incomplete responses and refractoriness to treatment in a significant fraction of the patients.

Immunosuppressors and biologics, while most of them are still in development for the treatment of NIU, appear to provide some additional help in the management of the more severe cases.

Nevertheless, despite these treatments, a large proportion of patients with uveitis are refractory to treatment and experienced reduction of vision.

As a consequence, NIU remains a significant unmet medical need and in this context, a new well-tolerated alternative, particularly with a new and multi-target, local mechanism of action through immunomodulation could represent a significant added value for the patients.

In the present invention, the Applicant aims to provide an alternative treatment for uveitis based on the use of Tr1 cells directed against an eye-associated antigen, such as, for example, type II collagen, which is one of the components of retina and vitreous humour.

In the art, immune response to IRBP and S-antigen were described in uveitis patients, but not to type II collagen. Surprisingly, the Inventors herein demonstrated that regulatory T cells directed to type II collagen may be used to treat uveitis, despite the apparent lack of immune response to this specific antigen in uveitis patients.

### SUMMARY OF THE INVENTION

The invention relates to a composition comprising at least one human regulatory T cell (Treg cell) population, preferably Tr1 cell population, directed against an eye-associated antigen.

In an embodiment, said human Treg cell population, preferably Tr1 cell population, is a human Tr1 clone population.

In an embodiment of the invention, said eye-associated antigen is selected from the group comprising type II collagen, retinal arrestin, S-arrestin, interphotoreceptor retinoid-binding proteins (IRBP1), betaB1-crystallin, retinal proteins, choroid proteins, and fragments, variants and mixtures thereof.

In a preferred embodiment, said eye-associated antigen is type II collagen and fragments, variants and mixtures thereof.

Another object of the invention is a medicament comprising or consisting of at least one human Treg cell population, preferably at least one human Tr1 cell population, directed against an eye-associated antigen.

Another object of the invention is a pharmaceutical composition comprising or consisting of at least one human Treg cell population, preferably at least one human Tr1 cell population directed against an eye-associated antigen in combination with one or more pharmaceutically acceptable carriers.

In one embodiment, said human Treg cell population, preferably Tr1 cell population, is a human Treg or Tr1 clone population.

In an embodiment of the invention, said medicament or pharmaceutical composition comprises at least one human Treg cell, preferably Tr1 cell, directed against an eye-associated antigen selected among type II collagen, retinal arrestin, S-arrestin, interphotoreceptor retinoid-binding proteins (IRBP1), betaB1-crystallin, retinal proteins, choroid proteins, and fragments, variants and mixtures thereof.

In a preferred embodiment, said human Treg cell, preferably Tr1 cell, is directed against type II collagen.

The invention relates to the use of a composition comprising or consisting of at least one human Treg cell population, preferably Tr1 cell population, directed against an eye-associated antigen for the preparation of a medicament or a pharmaceutical composition for treating uveitis.

In one embodiment of the invention, said eye-associated antigen is selected from the group comprising type II collagen, retinal arrestin, S-arrestin, interphotoreceptor retinoid-binding proteins (IRBP1), betaB1-crystallin, retinal proteins, choroid proteins, and fragments, variants and mixtures thereof.

In a preferred embodiment, said at least one human Treg cell population, preferably Tr1 cell population, is directed against type II collagen.

In one embodiment of the invention, the medicament or pharmaceutical composition that is to be administered to a subject in need thereof comprises human Treg cells, preferably Tr1 cells, autologous to the cells of said subject.

In a preferred embodiment, 10⁴ to 10⁷ Treg or Tr1 cells are administered to the subject in need thereof.

In one embodiment of the invention, the administration to said subject of an effective amount of the medicament or the pharmaceutical composition of the invention is in combination with one or more therapeutic agents used for treating uveitis.

In a preferred embodiment, said one or more therapeutic agents is a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

In one embodiment, the administration to said subject of an effective amount of Treg cells directed to an eye-associated antigen is in combination with human MSC.

In another embodiment of the invention, the medicament or the pharmaceutical composition of the invention is to be administrated to a subject that does not respond adequately to, or is unlikely to respond adequately to, a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

Another object of the invention is a method for treating uveitis in a subject in need thereof, comprising administering an effective amount of human Treg cells, preferably of human Tr1 cells, directed to an eye-associated antigen to the subject.

In one embodiment, said eye-associated antigen is selected from the group comprising type II collagen, retinal arrestin, S-arrestin, interphotoreceptor retinoid-binding proteins (IRBP1), betaB1-crystallin, retinal proteins, and choroid proteins, and fragments, variants and mixtures thereof. Preferably, said eye-associated antigen is type II collagen and fragments, variants and mixtures thereof.

In one embodiment, uveitis is non-infectious uveitis. In another embodiment, uveitis is chronic uveitis. In another embodiment, uveitis is recurrent uveitis.

In one embodiment, uveitis is panuveitis, or intermediate and/or posterior uveitis.

In one embodiment, the method of the invention is a method for treating macular oedema related to uveitis.

In one embodiment, human Treg cells or Tr1 cells are autologous to the cells of said subject.

In one embodiment, 10⁴ to 10⁷ Treg or Tr1 cells are administered to the subject in need thereof.

In one embodiment, the administration to said subject of an effective amount of Treg or Tr1 cells directed to an eye-associated antigen is in combination with one or more therapeutic agents used for treating uveitis.

In one embodiment, the administration to said subject of an effective amount of Treg cells, preferably Tr1 cells, directed to an eye-associated antigen is in combination with one or more therapeutic agents selected in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

In one embodiment, said subject does not respond adequately to, or is unlikely to respond adequately to, one or more therapeutic agents in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

Another object of the invention is a method for treating uveitis in a subject in need thereof, said method comprising the steps of:
obtaining Treg cells, preferably Tr1 cells, directed to a selected eye-associated antigen, said Treg or Tr1 cells being obtained from a blood sample of said subject,
further expanding Treg or Tr1 cells obtained at the previous step,
injecting Treg or Tr1 cells thus obtained in said subject, preferably by intravenous route.

Another object of the invention is a process for treating uvetitis in a subject in need thereof, said process comprising the steps of:
obtaining Treg cells, preferably Tr1 cells directed to a selected eye-associated antigen, said Treg or Tr1 cells being obtained from a blood sample of said subject,
cloning said Treg or Tr1 cells directed to a selected eye-associated antigen,
further expanding Treg or Tr1 clones obtained at the previous step,
re-injecting Treg or Tr1 clones thus obtained in said subject, preferably by intravenous route.

### BRIEF DESCRIPTION OF THE DRAWINGS

FIG. 1: IL-10 production of Tr1 clones after specific activation with Type II collagen. FIG. 1 describes the specific increase of the IL-10 production by T-cell clones in the presence of the specific antigen type II collagen. Clones were activated with or without type II collagen in the presence of irradiated autologous antigen presenting cells. After 48 hours, the IL-10 production was measured by ELISA.

FIG. 2: Cytokine secretion profile of type II collagen Tr1 clones. IL-10, IL-4 and IFNγ secretion of type II collagen specific Tr1 cell clones were measured in 48 hours supernatant of anti-CD3+anti-CD28 monoclonal antibodies activated cells.

FIG. 3: In vitro suppressive activity of type II collagen Tr1 clones. The suppressive activity of collagen type II Tr1 clones was evaluated in co-culture experiments with autologous CD4+ T lymphocytes. Cell populations were co-cultured during 3 days using anti-CD3+anti-CD28 monoclonal antibodies. Then, cell proliferation of the autologous CD4+ T cells was assessed in the absence or presence of graded quantities of Tr1 cells. Results show that the addition of Tr1 cells to CD4+ T lymphocytes massively inhibits T-cell proliferation.

FIG. 4: Surface expression of CD62L, CD127, CD39, GITR, CTLA-4 and intracellular expression of Granzyme B evaluated using flow cytometry on Tr1 cells directed to type II collagen from human (panel A, n=5) and from mice (panel B, n=5).

FIG. 5: Human (Panel A, n=4) and mouse Tr1 populations directed to type II collagen (Panel B, n=3) were activated in vitro using anti-CD3+anti-CD28 antibodies. Cytokine secretion (secretion of IL-10, IL-4, IL-13 and IFNγ) was then evaluated after 48 hours in the cell culture supernatants.

FIG. 6: Human T cells (Panel A, n=5) and mouse splenocytes (Panel B, n=4) have been activated using anti-CD3+anti-CD28 antibodies (human) or anti-CD3 alone (mice) and cultured with or without 48 hours supernatants of anti-CD3+anti-CD28 activated Tr1 cells directed to type II collagen. IL-17 secretion by activated T cells was measured after 7 days (human) or 48 hours (mouse). Human and mouse Tr1 cells directed to type II collagen cell supernatants show the capacity to suppress the pro-inflammatory cytokine production of CD3 activated T cells.

FIG. 7: Human T cells (Panel A, n=5) and mouse splenocytes (Panel B, n=4) have been activated using anti-CD3+anti-CD28 antibodies (human) or anti-CD3 alone (mice) and cultured with or without 48 hours supernatants of anti-CD3+anti-CD28 activated Tr1 cells directed to type II collagen. IFNγ secretion by activated T cells was measured after 7 days (human) or 48 hours (mouse). Human and mouse Tr1 cells directed to type II collagen cell supernatants show the capacity to suppress the pro-inflammatory cytokine production of CD3 activated T cells.

FIG. 8: Human (Panel A, n=5) and mouse (Panel B, n=4) Tr1 populations directed to type II collagen were assayed in an ATP hydrolysis assay. For both species, Tr1 cells directed to type II collagen show the capacity to release high levels of free inorganic phosphate (Pi) in the presence of ATP demonstrating an ATP hydrolysis capacity.

FIG. 9: Human (Panel A, n=2) and mouse (Panel B, n=4) Tr1 populations directed to type II collagen were tested in a 4 hour cytotoxic assay in co-culture with myeloid target cells. Results strongly indicate a cytotoxic activity of both human and mouse Tr1 cells directed to type II collagen.

FIGS. 10A and 10B: Clinical uveitis score has been evaluated at Day 15 in DBA-1 mice either non-immunized (naïve n=9), immunized and treated with saline (n=11) or immunized and administered intravenously with 3.10⁶ Tr1 cells directed to type II collagen 8 days after IRBP immunization (n=11). Panel A represents the clinical inflammatory score on the anterior part of the eye whereas Panel B represents the clinical intermediate (vitreous haze) and eye fundus findings assessed by ophthalmoscopy. For clinical evaluation, mice showing unobservable fundus linked to important iris synechiae were excluded from the analysis (4 in each group). ** p<0.01, single factor ANOVA statistical analysis.

FIGS. 10C and 10D: Histology score has been evaluated at Day 15 in DBA-1 mice either non-immunized (naïve n=6), immunized and treated with saline (n=6) or immunized and administered intravenously with 3.10⁶ Tr1 cells directed to type II collagen 8 days after IRBP (n=6). Panel C represents the infiltration of the eye (all locations) by proinflammatory cells and Panel D represents morphological findings on the retinal, choroid layers and on the anterior part of the eye.

FIGS. 10E, 10F, 10G and 1011: Representative eye histology of the EAU model. Panel E shows eye histology findings from IRBP immunized mice. Eye histology findings on naïve mice (Panel F), immunized mice treated with saline (Panel G) or with 3.10⁶ Tr1 cells directed to type II collagen (Panel H). Left panels objectives ×2, Right panel objectives ×10.

FIG. 11: Number of Tr1 cells in the lung, liver and eye 24 hours after intravenous injection of Tr1 cells to mice with (inflamed) or without (naïve) uveitis.

### DETAILED DESCRIPTION OF THE INVENTION

### Definition

The term “regulatory T cells (or Treg cells)” refers to cells that suppress, inhibit or prevent excessive or unwanted inflammatory responses, such as, for example, autoimmunity or allergic reactions. Regulatory T cells include:
thymus-derived Treg cells (tTreg, previously referred as “natural Treg cells”). As used herein, tTregs have the following phenotype at rest CD4⁺CD25⁺FoxP3⁺,
peripherally-derived Treg cells (pTreg, previously referred as “induced Treg cells”), including, for example, Tr1 cells, TGF-β secreting Th3 cells, regulatory NKT cells, regulatory γδT cells, regulatory CD8⁺ T cells, double negative regulatory T cells,
in vitro-induced Treg cells and
engineered Treg cells.

In one embodiment, the Treg cells of the invention, preferably the Tr1 cells of the invention, express at least three cytotoxic molecules. The term “cytotoxic molecules” refers to a protein involved in the cytotoxic pathway, preferably to a protein of the serine protease family or to the granzyme family or to granulyzin or to perforin. Preferably, the at least 3 cytotoxic molecules are selected from the group comprising granulysin, granzyme A, granzyme B, granzyme M, granzyme K, granzyme H and perforin.

In one embodiment, the at least three cytotoxic molecules comprise granzyme B. In one embodiment, the at least three cytotoxic molecules comprise granzyme B and granzyme H. In one embodiment, the Treg cell population of the invention is such that
at least 30% of the cells of the Treg population express granzyme B,
at least 80% of the cells of the Treg population express granzyme H, and
at least 30% of the cells of the Treg population express at least one cytotoxic molecule selected from granulysin, granzyme A, granzyme K, granzyme M and perforin.

The term “Tr1 cells” as used herein refers to cells having the following phenotype at rest CD4⁺CD25⁻CD127⁻ and the following phenotype when activated: CD4⁺CD25⁺CD127⁻. Tr1 cells, Type 1 T regulatory cells (Type 1 Treg) and IL-10 producing Treg are used herein with the same meaning. In one embodiment, Tr1 cells may be characterized, in part, by their unique cytokine profile: they produce IL-10, and IFN-gamma, but little or no IL-4 or IL-2. In one embodiment, Tr1 cells are also capable of producing IL-13 upon activation. The cytokine production is typically evaluated in cultures of cells after activation with polyclonal activators of T lymphocytes such as anti-CD3+anti-CD28 antibodies or Interleukin-2, PMA+ionomycin. Alternatively, the cytokine production is evaluated in cultures of cells after activation with the specific T-cell antigen presented by antigen presenting cells. Regarding production of IL-10, Tr1 cells may produce at least about 50 pg/ml, typically more than about 100, 500 pg/ml, or more than about 1, 2, 4, 6, 8, 10, 12, 14, 16, 18, or 20 thousand pg/ml or more. Regarding production of IFN-gamma, Tr1 cells may produce concentrations comprised between 0 pg/ml and at least 400 pg/ml, or of more than about 400 pg/mL, typically greater than about 600, 800, 1000, 1200, 1400, 1600, 1800, or 2000 pg/ml or more. Little or no IL-4 or IL-2 corresponds to less than about 1000 pg/ml, preferably less than 750, 500, 250, 100, 75, or 50 pg/ml, or less. Regarding the production of IL-13, Tr1 cells may produce at least about 1 ng/ml, typically at least about 10, 25, 50, or 80 ng/ml.

The term “Th3 cells” as used herein refers to cells having the following phenotype CD4⁺FoxP3⁺ and capable of secreting high levels TGF-β upon activation, low amounts of IL-4 and IL-10 and no IFN-γ or IL-2. These cells are TGF-β derived.

The term “regulatory NKT cells” as used herein refers to cells having the following phenotype at rest CD161⁺CD56⁺CD16⁺ and expressing a Vα24/Vβ11 TCR.

The term “regulatory CD8⁺ T cells” as used herein refers to cells having the following phenotype at rest CD8⁺CD122⁺ and capable of secreting high levels of IL-10 upon activation.

The term “double negative regulatory T cells” as used herein refers to cells having the following phenotype at rest TCRαβ⁺CD4⁻CD8⁻.

The term “in vitro inducible regulatory T cells” as used herein refers to naive T cells that are differentiated into regulatory T cells in vitro. One example of said in vitro inducible regulatory T cells is Th3 cells that are differentiated from naïve T cells in the presence of TGF-β. Other examples are natural regulatory T cells or Tr1 cells obtained by in vitro differentiation.

The term “γδ T cells” as used herein refers to T lymphocytes that express the [gamma][delta] heterodimer of the TCR. Unlike the [alpha][beta] T lymphocytes, they recognize non-peptide antigens via a mechanism independent of presentation by MHC molecules. Two populations of γδT cells may be described: the γδT lymphocytes with the Vγ9Vδ2 receptor, which represent the majority population in peripheral blood and the γδT lymphocytes with the Vδ1 receptor, which represent the majority population in the mucosa and have only a very limited presence in peripheral blood. Vγ9Vδ2 T lymphocytes are known to be involved in the immune response against intracellular pathogens and hematological diseases.

The term “engineered Treg cells” as used herein refers to T lymphocytes genetically engineered, such as, for example T lymphocytes wherein the expression of transgenes has been induced, thereby obtaining regulatory T cells. This term also encompasses Treg cell populations that have been modified by introduction of transgenes or deletion or inhibition of expression or translation of endogenous genes.

The term “antigen” as used herein refers to a protein, or peptide for which the cells of this invention are specific. In one embodiment, the term “antigen” may refer to a synthetically derived molecule, or a naturally derived molecule, which shares sequence homology with an antigen of interest, or structural homology with an antigen of interest, or a combination thereof. In one embodiment, the antigen may be a mimetope. A “fragment” of the antigen refers to any subset of the antigen, as a shorter peptide. A “variant” of the antigen refers to a molecule substantially similar to either the entire antigen or a fragment thereof. Variant antigens may be conveniently prepared by direct chemical synthesis of the variant peptide, using methods well-known in the art.

The term “subject” as used herein refers to a mammal, preferably to a human being. In one embodiment, the subject is not affected with or diagnosed with an arthritic condition, such as, for example, rheumatoid arthritis.

The term “effective amount” as used herein refers to an amount sufficient to cause a beneficial or desired clinical result (e.g. improvement in clinical condition).

The term “clone” or “clone population” as used herein refers to a population of differentiated cells being derived from a unique differentiated cell.

The term “treatment” or “treating” as used herein generally refers to a clinical intervention in an attempt to alter the natural course of the individual being treated, and may be performed during the course of clinical pathology. Desirable effects include, but are not limited to, alleviating symptoms, suppressing, diminishing or inhibiting any direct or indirect pathological consequences of the disease, lowering the rate of disease progression, ameliorating or palliating the disease state, and causing remission or improved prognosis.

In the context of the invention, it refers to any improvement in the clinical symptoms of uveitis, as well as any improvement in the well-being of the patients, in particular an improvement manifested by at least one of the following: reduction of visual haze, maintenance or improvement of vision, therapeutic response that may be assessed by dilated fundus examination or by other assessment method aiming at observing for example, healing or decrease of inflammatory lesions, tissue destruction, biomarkers of autoimmunity and/or inflammation, vasculitis, disruption of the retina blood bather, cellular infiltration, oedema, or renewal of tissues, presence of retinal folds or retinal detachment.

In one embodiment, treatment of uveitis may correspond to reduction of the grade for anterior chamber cells, as described in Zierhut et al (European ophthalmic review, 2007). In this grading scheme, the number of cells in a field having a size of 1×1 mm slit beam is measured, and grade O corresponds to <1 cells in field, grade 0.5+ corresponds to 1-5 cells in field, grade 1+ corresponds to 6-15 cells in field, grade 2+ corresponds to 16-25 cells in field, grade 3+ corresponds to 26-50 cells in field, and grade 4+ corresponds to >50 cells in field.

In another embodiment, treatment of uveitis may correspond to reduction of the grade for anterior chamber flare, as described in Zierhut et al (European ophthalmic review, 2007). In this grading scheme, the flair is evaluated, with grade 0 corresponding to the absence of flare, grade 1+ corresponding to faint flare, grade 2+ corresponding to moderate flare (iris and lens details clear), grade 3+ corresponding to marked flare (iris and lens details hazy), and grade 4+ corresponding to intense flare (fibrin or plastic aqueous).

In one embodiment, treatment of uveitis may correspond to reduction of vitreous haze, as described in Zierhut et al (European ophthalmic review, 2007). In this grading scheme, vitreous haze is analysed, with score 0 (Nil) corresponding to no clinical finding, score 1 (minimal) corresponding to a posterior pole clearly visible, score 2 (mild) corresponding to posterior pole details slightly hazy, score 3 (moderate) corresponding to posterior pole details very hazy, score 4 (marked) corresponding to posterior pole details barely visible and score 5 (severe) corresponding to a stage where fundal details are not visible.

In one embodiment, maintenance or improvement of vision may be assessed by scores of visual acuity, which are well known by the skilled artisan.

The term “uveitis” as used herein refers to inflammation of the eye that may affect the uvea, or middle layer of the eye but also the lens, retina, optic nerve, and vitreous chamber. Uveitis may involve the full eye (panuveitis) or a segment of the eye (anterior, intermediate or posterior). Examples of uveitis include, but are not limited to, anterior uveitis (comprising iritis, iridiocyclitis, and anterior cylitis), intermediate uveitis (comprising pars planitis, posterior cyclitis, and hyalitis), posterior uveitis (comprising focal, multifocal or diffuse choroiditis, chorioretinitis, retinochoroiditis, retinitis, and neuroretinitis), panuveitis, acute uveitis, recurring uveitis and chronic uveitis. In one embodiment, uveitis is non-infectious uveitis. Examples of causes of non-infectious uveitis include, but are not limited to systemic autoimmune disorders (such as, for example Behcet's disease and Vogt-Koyanagi-Harada (VKH) disease); trauma and surgery. In one embodiment, non-infectious uveitis is idiopathic non-infectious uveitis.

The term “about” preceding a figure means plus or less 10% of the value of said figure.

The Present Invention

The present invention relates to a method for treating uveitis in a subject in need thereof, comprising the administration to said subject of a composition comprising or consisting of human Treg cells directed against an eye-associated antigen, preferably human Tr1 cells directed against an eye-associated antigen.

In one embodiment, the method of the invention further comprises a step of monitoring the treatment of uveitis in the subject, wherein said monitoring may correspond to the assessment of the reduction of visual haze or of the maintenance or improvement of vision, or to the determination of a therapeutic response that may be assessed by dilated fundus examination or by other assessment method aiming at observing for example, healing or decrease of inflammatory lesions, tissue destruction, biomarkers of autoimmunity and/or inflammation, vasculitis, disruption of the retina blood barrier, cellular infiltration, oedema, or renewal of tissues, presence of retinal folds or retinal detachment.

According to the invention, the “human Tr1 cell population” corresponds to Tr1 cells as described here above in the definitions and does not include CD4+CD25+ regulatory T cells or FoxP3+ regulatory T cells (natural or conventional Treg), TGF-β secreting Th3 cells, or regulatory NKT cells.

According to the invention, the term “eye-associated antigen” refers to an immunogenic peptide, which is present in the eye.

In one embodiment of the invention, said immunogenic peptide may be present in the resting eye.

In another embodiment, said immunogenic peptide may be present in an inflammatory eye. Examples of eye-associated antigens include, but are not limited to, type II collagen, retinal arrestin, S-arrestin, interphotoreceptor retinoid-binding proteins (IRBP1), betaB1-crystallin, retinal proteins, and choroid proteins.

In a preferred embodiment, said human Treg cells, preferably said Tr1 cells are directed against type II collagen.

Human Treg or Tr1 cells directed against type II collagen may be directed against epitopes present in the 245-273 fragment of type II collagen (IAGAPGFPGPRGPPGPQGATGPLGPKGQT, SEQ ID NO: 1) and associated either with HLA-DR1 or HLA-DR4 subjects.

Without whishing to be bound to a theory, the Applicant assume that the injected Treg cell population, preferably said Tr1 cell population, directed to an eye-associated antigen would be activated in vivo by the antigen present in the eye and then would be able to control uveitis. There is therefore no need of injection of the antigen to which the Treg or Tr1 cells are directed to stimulate these cells.

Therefore, in one embodiment, the method of the invention for treating uveitis comprises:
the administration of regulatory T cells directed to an eye-associated antigen to an uveitis patient,
the migration of the administered regulatory T cells to the eye (i.e. to the inflammation site),
the activation in vivo of the administered regulatory T cells by the antigen present in the eye (wherein said antigen is presented by antigen presenting cells of the eye), and
the control of inflammation by the administered regulatory T cells.

The control of inflammation by the administered regulatory T cells in the case of uveitis occurs only in the eye and not in an associated lymphoid organs, such as, for example, lymph nodes, as there is no lymphoid organs associated to the eye.

The present invention also relates to a composition comprising or consisting of at least one human Treg cell population directed against an eye-associated antigen, preferably at least one human Tr1 cell population directed against an eye-associated antigen.

In a preferred embodiment, said composition comprises or consists of a human Treg cell population directed against type II collagen, preferably human Tr1 cell population directed against type II collagen.

In one embodiment of the invention, human Tr1 cells may be obtained by
a) isolating a progenitor cell population from a subject,
b) obtaining a population of dendritic cells by culturing said progenitor cell population in the presence of IL-10,
c) contacting cells of step b) with a CD4+ T lymphocyte population isolated from said subject in the presence of an eye-associated antigen to allow differentiation of CD4+ T cells directed to said antigen into the Tr1 cell population, and
d) recovering the Tr1 cell population from the step c).

In step b), IL-10 is present from 50 to 250 U/ml, preferably at 100 U/ml in the culture medium. Said method for obtaining Tr1 cells is described in Wakkach et al (Immunity 2003 May; 18(5):605-17).

Said method may also be carried out using Dexamethasone and Vitamin D3, or tolerogenised or immature DCs instead of the DCs of step b).

In another embodiment of the present invention, human Tr1 cells may be obtained by:

a) culturing a CD4+ T cell population directed to an eye-associated antigen isolated from a subject in a media with an appropriate amount of IFN-α, and

b) recovering the Tr1 cell population.

IFN-α is preferably present in the media at 5 ng/ml. In step a), the media may further comprise an appropriate amount of IL-10, preferably at 100 U/ml.

In step b), the Tr1 cell population is cultured in a media comprising IL-15 to allow proliferation, IL-15 being preferably at 5 ng/ml in the media. Said method for obtaining Tr1 cells is described in the patent U.S. Pat. No. 6,746,670.

In still another embodiment of the invention, human Tr1 cells may be obtained by:

a) in vitro activating a CD4+ T cell population in presence of an eye-associated antigen, presented by artificial antigen presenting cells, and b) recovering an activated CD4+ T cells comprising at least 10% of Tr1 cells.

Preferably, the artificial antigen presenting cells express a HLA II system molecule and a human LFA-3 molecule and do not express the co-stimulation molecules B7-1, B7-2, B7-H1, CD40, CD23 and ICAM-1.

Said process, for obtaining Tr1 cells is described in the patent application WO02/092793.

In still another embodiment of the invention, human Tr1 cells may be obtained by:

a) in vitro activating a CD4+ T cell population in the presence of an eye-associated antigen and an appropriate amount of IL-10; and

b) recovering the Tr1 cell population.

Preferably, IL-10 is present in the media at 100 U/ml. Said method is described in Groux et al. (Nature 1997, 389(6652):737-42).

In still another embodiment of the invention, human Tr1 cells may be obtained by:

a) stimulating a leukocyte population or a peripheral blood mononuclear cell (PBMC) population with an eye-associated antigen,

b) recovering the antigen-specific Tr1 cell population from the stimulated population,

c) optionally expanding said antigen-specific Tr1 cell population.

Leukocytes encompass several types of cells, which are characterized by their importance, their distribution, their number, their lifetime and their potentiality. These types are the following: the polynuclear or granular leukocytes, among which one finds the eosinophilic, the neutrophilic and the basophilic leukocytes, and the mononuclear cells, or peripheral blood mononuclear cells (PBMCs), which are large white blood cells and consist in the major cell types of the immune system (lymphocytes and monocytes). The leukocytes or the PBMCs can be separated from the peripheral blood by any method known to those skilled in the art. Advantageously, for the separation of the PBMCs, centrifugation may be used, preferably density gradient centrifugation, preferably discontinuous density gradient centrifugation. An alternative is the use of specific monoclonal antibodies. In certain embodiments PBMC are typically isolated from the whole blood product by means of Ficoll-Hypaque, using standard procedures. In other embodiments the PBMCs are recovered by means of leukapheresis.

Said method is described in the patent application WO2007/010406.

In still another embodiment, human Tr1 cells may be obtained by:

a) culturing a leukocyte population or a peripheral blood mononuclear cell (PBMC) population with mesenchymal stem cells in the presence of an eye-associated antigen,

b) recovering the Tr1 cell population.

Said method can also be carried out with naïve or memory T cells instead of PBMC or leukocytes.

The Tr1 cell population thus obtained may further be expanded by culture in presence of cytokines such as Interleukin-2 and Interleukin-4. Alternatively, Interleukin-15 and Interleukin-13 could also be used in Tr1 cell expansion cultures.

In the methods described above, human Tr1 cells can be characterized by the identification method described in WO2005/000344. Said identification method of Tr1 cells is based on the detection of the simultaneous presence of expression products of genes coding CD4 molecule and molecules from the group comprising CD18 and/or CD11a, and CD49b. Tr1 cells can be identified and/or purified by Elisa, flow cytometry, or immunoaffinity methods with antibodies directed against said markers.

In another embodiment, human Tr1 cells can be characterized by the identification method described in WO2011/128779. Said identification method of Tr1 cells is based on the detection of the expression or absence of expression of CD4, CD62L and CD127. Tr1 cells can be identified and/or purified by Elisa, flow cytometry, or immuno-affinity methods with antibodies directed against said markers.

Tr1 cells can also be enriched by positive selection or negative selection using flow cytometry or magnetic beads or immuno-affinity methods. Such methods are also described in WO2005/000344.

Examples of methods for expanding Tr1 cells are well known to the skilled artisan, and include, but are not limited to:
use of stimulatory antibodies, commonly anti-CD3 and anti-CD28, absorbed on magnetic beads or nanoparticles and use of stimulatory cytokines (commonly from the group of IL-2, IL-15 and IL-4)
use of stimulatory antibodies, commonly anti-CD3 and anti-CD28, and use of ligands specific for the stimulatory antibodies absorbed on magnetic beads or nanoparticles and use of stimulatory cytokines (commonly from the group of IL-2, IL-15 and IL-4)
use of stimulatory antibodies, commonly anti-CD3 and anti-CD28, coated on a surface of a cell culture vessel and use of stimulatory cytokines (commonly from the group of IL-2, IL-15 and IL-4), and
use of stimulatory antibodies, commonly anti-CD3 coated on a surface of a cell culture vessel and soluble anti-CD28 and use of stimulatory cytokines (commonly from the group of IL-2, IL-15 and IL-4).

In another embodiment of the present invention, the Tr1 cells directed to an eye-associated antigen may be expanded by the in vitro method described in WO2006/108882. Said method comprises:

a) cultivating at a temperature T1 inferior to 35° C., in a culture medium Mf, feeder cells such as insect feeder cells, said temperature T1 allowing the proliferation of feeder cells and said feeder cells expressing factors which interact with the following cell surface proteins:
the CD3/TCR complex,
the CD28 protein,
the IL-2 receptor,
the CD2 protein,
the IL-4 receptor,

b) contacting the feeder cells obtained in step a) cleared or not of their culture medium Mf, with the Tr1 cell population contained in the culture medium Mp, wherein said culture medium Mp does not initially contain the factors cited in step a), in order to obtain a mixture containing the Tr1 cell population, the feeder cells and the culture medium Mp,

c) cultivating the mixture obtained at step b) at a temperature T2 which is at least 35° C., said temperature being chosen such that the Tr1 cell population proliferates and the feeder cells do not proliferate,

d) recovering the Tr1 cell population such expanded.

Examples of factors which interact with the above mentioned cell surface proteins include:
a modified anti-CD3 antibody, wherein the anti-CD3 intracytoplasmic domain of the CD3 heavy chain is replaced with a transmembrane domain,
the CD80 or CD86 protein,
the IL-2 secreted by the feeder cells,
the CD58 protein,
an interleukin selected from the group comprising IL-4 and IL-13.

In a preferred embodiment of the present invention, said Treg or Tr1 cells directed to an eye-associated antigen may be cloned by using conventional methods for cloning T cells.

In preferred embodiment of the present invention, said composition comprising at least one human Treg or Tr1 cell population directed against an eye-associated antigen or at least one clone of human Treg or Tr1 cell directed against an eye-associated antigen may be frozen to be stored.

In a preferred embodiment of the present invention, said eye-associated antigen is selected from the group comprising type II collagen, retinal arrestin, S-arrestin, interphotoreceptor retinoid-binding proteins (IRBP1), betaB1-crystallin, retinal proteins, and choroid proteins, fragments, variants and mixtures thereof. Preferably, the eye-associated antigen is a recombinant or a synthesized antigen.

Preferably, said eye-associated antigen is type II collagen, fragments, variants and mixtures thereof.

The term “variant” of the eye-associated antigen refers herein to an antigen that is almost identical to the natural antigen and which shares the same biological activity. The minimal difference between the natural antigen and its variants may lie for example in an amino-acid substitution, deletion, and/or addition. Such variants may contain for example conservative amino acid substitutions in which amino acid residues are replaced with amino acid residues having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Another object of the present invention is to provide a medicament comprising or consisting of at least one human Treg cell population directed against an eye-associated antigen, preferably at least one human Tr1 cell population directed against an eye-associated antigen.

The present invention also intends to provide a pharmaceutical composition comprising or consisting of at least one human Treg, preferably Tr1 cell population directed against an eye-associated antigen in combination with one or more pharmaceutically acceptable carriers.

According to one embodiment, said human Treg, preferably Tr1 cell population is a human Treg or Tr1 clone population.

According to a preferred embodiment, said joint-associated antigen is selected among type II collagen, retinal arrestin, S-arrestin, interphotoreceptor retinoid-binding proteins (IRBP1), betaB1-crystallin, retinal proteins, and choroid proteins, and fragments, variants and mixtures thereof.

According to a more preferred embodiment, the medicament or the pharmaceutical composition of the invention comprises at least one human Treg cell population or clone directed against type II collagen, preferably at least one human Tr1 cell population or clone directed against type II collagen.

Human Treg or Tr1 cells directed against type II collagen may be directed against epitopes present in the 245-273 fragment of type II collagen (IAGAPGFPGPRGPPGPQGATGPLGPKGQT, SEQ ID NO 1) and associated either with HLA-DR1 or HLA-DR4 subjects.

The pharmaceutically acceptable carriers useful herein are conventional. Remington's Pharmaceutical Sciences 16^{th} edition, Osol, A. Ed. (1980) describes compositions and formulations suitable for pharmaceutical delivery of the composition of the present invention. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as vehicle. The carrier and composition can be sterile, and the formulation suits the mode of administration. In addition to biological neutral carriers, pharmaceutical compositions to be administrated can contain minor amounts of non-toxic auxiliary substances, such as wetting or emulsifying agents, preservatives, and pH buffering agents and the like, for example sodium acetate or sorbitan monolaurate. The composition can be a liquid solution, suspension, emulsion.

The present invention relates to the use of a composition comprising at least one human Treg cell population directed against an eye-associated antigen, preferably at least one human Tr1 cell population directed against an eye-associated antigen for the preparation of a medicament or a pharmaceutical composition for treating uveitis.

An object of the invention is the medicament or pharmaceutical composition as described here above for treating uveitis or for use in treating uveitis.

Preferably, uveitis is non-infectious uveitis. Examples of causes of non-infectious uveitis include, but are not limited to systemic autoimmune disorders (such as, for example Behcet's disease and Vogt-Koyanagi-Harada (VKH) disease); trauma and surgery. In one embodiment, non-infectious uveitis is idiopathic non-infectious uveitis.

Examples of uveitis include, but are not limited to, anterior uveitis (comprising iritis, iridiocyclitis, and anterior cylitis), intermediate uveitis (comprising pars planitis, posterior cyclitis, and hyalitis), posterior uveitis (comprising choroiditis, chorioretinitis, retinochoroiditis, retinitis, and neuroretinitis), panuveitis, acute uveitis, recurring uveitis and chronic uveitis

In one embodiment, uveitis is chronic or acute uveitis, preferably chronic uveitis. In one embodiment, uveitis is recurrent uveitis.

In one embodiment, uveitis is panuveitis or uveitis affecting the intermediate and/or posterior segment(s) of the eye (i.e. intermediate and/or posterior uveitis). In another embodiment, uveitis affects the anterior segment of the eye (i.e. anterior uveitis).

In one embodiment, the method of the invention is for treating comorbidities of uveitis, including cataracts, glaucoma, retinal vascular ischemia and macular oedema.

According to one embodiment, said human Treg, preferably Tr1 cell population is a human Treg or Tr1 clone population.

According to one embodiment, said one human Treg or Tr1 cell population or clone is directed against an eye-associated antigen selected among type II collagen, retinal arrestin, S-arrestin, interphotoreceptor retinoid-binding proteins (IRBP1), betaB1-crystallin, retinal proteins, and choroid proteins, and fragments, variants and mixtures thereof.

According to a more preferred embodiment, said at least one human Treg or Tr1 cell population or clone is directed against type II collagen.

Human Treg or Tr1 cells or clones directed against type II collagen may be directed against epitopes present in the 245-273 fragment of type II collagen (IAGAPGFPGPRGPPGPQGATGPLGPKGQT, SEQ ID NO 1) and associated either with HLA-DR1 or HLA-DR4 subjects.

An object of the present invention is also a method for treating uveitis in a subject in need thereof, comprising administering to said subject an effective amount of a medicament as described here above or a pharmaceutical composition as described here above.

Preferably, uveitis is non-infectious uveitis. Examples of causes of non-infectious uveitis include, but are not limited to systemic autoimmune disorders (such as, for example Behcet's disease and Vogt-Koyanagi-Harada (VKH) disease); trauma and surgery. In one embodiment, non-infectious uveitis is idiopathic non-infectious uveitis.

Examples of uveitis include, but are not limited to, anterior uveitis (comprising iritis, iridiocyclitis, and anterior cylitis), intermediate uveitis (comprising pars planitis, posterior cyclitis, and hyalitis), posterior uveitis (comprising choroiditis, chorioretinitis, retinochoroiditis, retinitis, and neuroretinitis), panuveitis, acute uveitis, recurring uveitis and chronic uveitis. In one embodiment, uveitis is chronic or acute uveitis, preferably chronic uveitis. In one embodiment, uveitis is recurrent uveitis.

In one embodiment, uveitis is panuveitis or uveitis affecting the intermediate and/or posterior segment(s) of the eye (i.e. intermediate and/or posterior uveitis). In another embodiment, uveitis affects the anterior segment of the eye (i.e. anterior uveitis).

In one embodiment, the method of the invention is for treating comorbidities of uveitis, including cataracts, glaucoma, retinal vascular ischemia and macular oedema.

The composition may be formulated for parenteral, intramuscular, intravenous, intraperitoneal, injection, intranasal inhalation, lung inhalation, intradermal, intra-articular, intrathecal injection. In one embodiment of the invention, the medicament or pharmaceutical composition of the invention may be injected within the eye to be treated, such as, for example, by intravitreal injection.

Preferably, the medicament or pharmaceutical composition of the invention may be administrated by intra-articular, intraperitoneal or intravenous injection, or by direct injection into the lymph nodes of the patient, preferably by intravenous injection.

In one embodiment, the medicament or pharmaceutical composition is administered by intravenous infusion, such as, for example, at an infusion rate ranging from about 1 mL/min to about 50 mL/min, preferably at an infusion rate of about 10 mL/min

In one embodiment, the medicament of pharmaceutical composition is in a form adapted for injection, such as, for example, a suspension (e.g. a sterile aqueous suspension), a dispersion, a solution or an emulsion. In one embodiment, the medicament of pharmaceutical composition is a cell suspension in human-serum albumin

The amount of Treg cells, preferably of Tr1 cells, directed to an eye-associated antigen effective in the treatment of uveitis will depend on the nature of uveitis, and can be determined by standard clinical techniques. The precise dose to be employed in the formulation will also depend on the route of administration and the seriousness of the disease or disorder, and should be decided according to the judgment of the practitioner and each individual's circumstances. Effective doses can be extrapolated from dose-response curves derived from in vitro or animal model test systems.

In one embodiment of the present invention, 10⁴/kg to 10⁹/kg cells are administrated to the subject. Preferably 10⁵/kg to 10⁷/kg cells and more preferably about 10⁶/kg cells are administrated to the subject.

In one embodiment of the present invention, from about 10⁴ to about 10⁷ cells, preferably from about 10⁶ to about 10⁷ cells are administrated to the subject. In one embodiment, about 10⁴, 10⁵, 10⁶, 10⁷, or 10⁸ cells are administered to the subject.

In one embodiment of the invention, the subject is administrated with the medicament at the time when flare-up are demonstrated by a decline in the clinical status of the subject or at the time when inflammatory lesions can be visualized for example by dilated fundus examination.

In one embodiment of the invention, the subject is administrated once with the medicament or the pharmaceutical composition of the present invention.

In a second embodiment of the invention, the subject is administrated once a month with the medicament or the pharmaceutical composition of the present invention.

In a third embodiment of the invention, the subject is administrated once a quarter with the medicament or the pharmaceutical composition of the present invention.

In a fourth embodiment of the invention, the subject is administrated once to twice a year with the medicament or the pharmaceutical composition of the present invention.

In one embodiment, the subject is administered once every 1, 2, 3 or 4 weeks with the medicament or the pharmaceutical composition of the present invention, or once every 6, 7, 8, 9, 10, 11 or 12 weeks with the medicament or the pharmaceutical composition of the present invention.

In another embodiment of the present invention, the medicament or pharmaceutical composition to be administered to a subject in need thereof comprises human Treg cells, preferably Tr1 cells, autologous to the cells of said subject.

This means that Treg or Tr1 cells will be administrated to the subject they come from or that precursors used for the production of Treg or Tr1 cells come from the subject the Treg or Tr1 cells will be administrated to.

In another embodiment of the present invention, the medicament or pharmaceutical composition to be administered to a subject in need thereof comprises human Treg, preferably Tr1 cells allogeneic to the cells of said subject. The term “allogeneic cells” as used herein refers to cells isolated from one subject (the donor) and infused in another patient (the recipient or host).

The present invention relates also to a process for treating uveitis in a subject in need thereof, said process comprising the steps of:
collecting a blood sample of said subject,
obtaining Treg, preferably Tr1, cells directed to a selected eye-associated antigen,
cloning said Treg or Tr1 cells directed to a selected eye-associated antigen,
further expanding Treg or Tr1 clones obtained at the previous step,
injecting Treg or Tr1 clones thus obtained in said subject, preferably by intravenous route.

The present invention relates also to a process for treating uveitis in a subject in need thereof, said process comprising the steps of:
collecting a blood sample of said subject,
obtaining Treg, preferably Tr1, cells directed to a selected eye-associated antigen,
further expanding Treg or Tr1 cells obtained at the previous step,
injecting Treg or Tr1 cells thus obtained in said subject, preferably by intravenous route.

Preferably, cloning and expansion of Tr1 clones or cells directed to a selected eye-associated antigen is carried out with the following method:

a) cultivating at a temperature T1 inferior to 35° C., in a culture medium Mf, feeder cells such as insect feeder cells, said temperature T1 allowing the proliferation of feeder cells and said feeder cells expressing factors which interact with the following cell surface proteins:
the CD3/TCR complex,
the CD28 protein,
the IL-2 receptor,
the CD2 protein,
the IL-4 receptor,

b) contacting the feeder cells obtained in step a) cleared or not of their culture medium Mf, with the Tr1 cell population contained in the culture medium Mp, wherein said culture medium Mp does not initially contain the factors cited in step a), in order to obtain a mixture containing the Tr1 cell population, the feeder cells and the culture medium Mp,

c) cultivating the mixture obtained at step b) at a temperature T2 which is at least 35° C., said temperature being chosen such that the Tr1 cell population proliferates and the feeder cells do not proliferate,

d) recovering the Tr1 cell population such expanded.

Examples of factors which interact with the above mentioned cell surface proteins include:
a modified anti-CD3 antibody, wherein the anti-CD3 intracytoplasmic domain of the CD3 heavy chain is replaced with a transmembrane domain,
the CD80 or CD86 protein,
the IL-2 secreted by the feeder cells,
the CD58 protein,
an interleukin selected from the group comprising IL-4 and IL-13.

In another embodiment of the present invention, the method for treating uveitis in a subject in need thereof comprises the administration to said subject of an effective amount of the medicament or the pharmaceutical composition of the invention in combination with one or more therapeutic agents used for treating uveitis.

The present invention relates to the use of the pharmaceutical composition or medicament of the invention, wherein the administration to said subject of an effective amount of the medicament or the pharmaceutical composition of the invention is in combination with one or more therapeutic agents used for treating uveitis.

Examples of therapeutic agents commonly used for treating uveitis are the following: a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

In a preferred embodiment of the present invention, the method for treating uveitis in a subject in need thereof comprises the administration to said subject of an effective amount of the medicament or the pharmaceutical composition of the invention in combination with one or more therapeutic agents selected in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

Preferably, the method for treating uveitis in a subject in need thereof comprises the administration to said subject of an effective amount of the medicament or the pharmaceutical composition of the invention in combination with a steroid.

In one embodiment, the one or more additional therapeutic agents used in combination with Tr1 cells are administered by systemic route (such as, for example, by injection, including intravenous injection), or locally to the eye (such as, for example, by intravitreal injection or using a retinal implant).

In one embodiment, the method for treating uveitis in a subject in need thereof comprises the administration to said subject of an effective amount of the medicament or the pharmaceutical composition of the invention in combination with human mesenchymal stem cells (MSCs).

In one embodiment of the invention, MSCs may be obtained by harvesting a MSCs-containing tissue and isolating and expanding said MSCs.

The MSCs obtained may be a homogeneous population or may be a mixed cell population enriched in MSCs. Homogeneous MSCs may be obtained by culturing adherent marrow or periosteal cells, or stroma-vascular fraction of adipose tissue and the MSCs may be identified by specific cell surface markers. The homogeneous MSC compositions may be obtained by positive selection of adherent marrow, stroma-vascular fraction of adipose tissue or periosteal cells which are free of markers associated with either hematopoietic cell or differentiated mesenchymal cells. These isolated mesenchymal cell populations display epitopic characteristics associated with only mesenchymal stem cells, have the ability to regenerate in culture without differentiating, and have the ability to differentiate into specific mesenchymal lineages when either induced in vitro or placed in vivo at the site of damaged tissue. In order to obtain subject human mesenchymal stem cells, it is necessary to isolate rare pluripotent mesenchymal stem cells from other cells in the bone marrow or other MSC source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, blood and adipose tissues.

A method, incorporated herewith by reference, for obtaining a cell population enriched in MSCs is described for example in the patent U.S. Pat. No. 5,486,359.

In a preferred embodiment of the invention, said Treg cells (preferably said Tr1 cells) and MSCs are autologous. This means that MSCs and Treg or Tr1 cells or precursors thereof are obtained from the same subject and will be administrated to the subject they come from.

The present invention relates to the use of the pharmaceutical composition or medicament of the invention for treating uveitis in a subject in need thereof, wherein the administration to said subject of an effective amount of the medicament or the pharmaceutical composition of the invention is in combination with one or more therapeutic agents selected in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

Preferably, the administration of an effective amount of the medicament or the pharmaceutical composition of the invention is in combination with a steroid.

In one embodiment, the administration of an effective amount of the medicament or the pharmaceutical composition of the invention is in combination with human MSC.

In another embodiment, the present invention also relates to a method of treatment of uveitis in which the medicament or the pharmaceutical composition of the invention is to be administrated to a subject in need thereof, wherein the subject does not respond adequately to, or is unlikely to respond adequately to, one or more therapeutic agents in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

The present invention relates to the use of the pharmaceutical composition or medicament of the invention, wherein said subject does not respond adequately to, or is unlikely to respond adequately to, one or more therapeutic agents in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

“Inadequate response”, “does not respond adequately to”, or “unlikely to respond adequately” refer to an actual or probable response by a subject which indicates that the therapy has been, or is likely to be, ineffective, toxic, or poorly tolerated insofar as the subject is concerned.

### EXAMPLES

In the following description, all experiments for which no detailed protocol is given are performed according to standard protocol.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

Tr1 Cell Isolation

Blood samples from healthy patients were collected and white blood cells were separated using gradient density centrifugation. Cells were then cultured in the presence of type II collagen in order to induce the specific proliferation of Tr1 cells directed against this antigen. After 13 days of culture, cell populations were cloned by limiting dilution method. Clones were then assessed for their specificity to type II collagen and for characteristic Tr1 cytokine production profile.

Cytokine Assays

For the determination of antigen specificity, sandwich ELISAs were performed on 48 hours supernatants of T-cell clones stimulated in the presence of antigen presenting cells (4.10⁵) and in the presence or absence of the specific antigen (type II collagen). For the determination of the cytokine production profile, type II collagen Tr1 cell clones were stimulated with anti-CD3+anti-CD28 monoclonal antibodies and the supernatants were harvested after 48 hours. ELISAs were performed using anti-IL-4 (11B11), anti-IL-10 (2A5), anti-IFN-γ (XGM1.2), biotin anti-IL-4 (24G2), anti-IL-10 (SXC1), anti-IFN-γ (R4-6A2) (Pharmingen Becton Dickinson).

Suppression Studies

For suppression studies, graded quantities of type II collagen specific Tr1 clones were co-cultured with autologous CD4 positive T lymphocytes. Co-cultures were stimulated with anti-CD3+anti-CD28 monoclonal antibodies. Alternatively, supernatants from collagen II specific clones were added to CD4 positive T lymphocytes stimulated with anti-CD3+anti-CD28 monoclonal antibodies. After 3 days total cell proliferation was assessed using the WST-1 proliferation kit from Roche.

Results

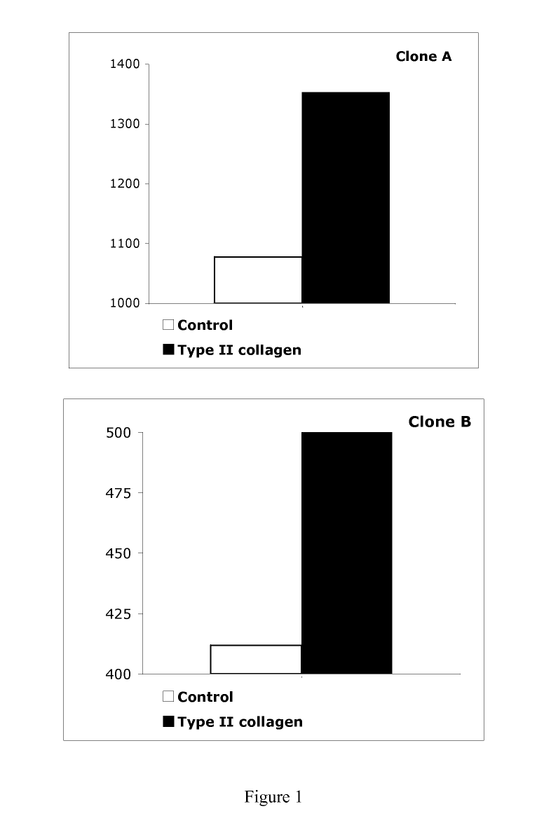

FIG. 1 shows the IL-10 production of two distinct Tr1 cell populations specific for type II collagen in the presence or absence of the antigen. Results show that type II collagen stimulation induces an increase in the production of IL-10. These results demonstrate the specificity of the cell populations toward type II collagen.

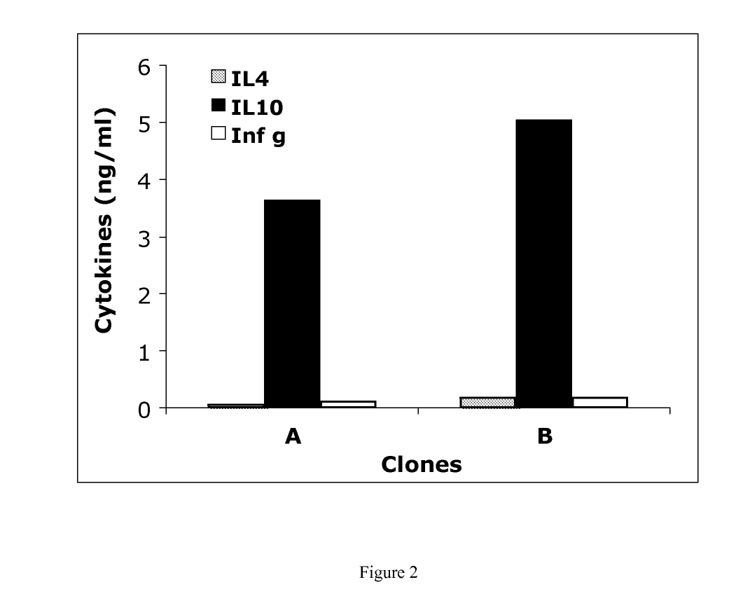

To further determine the cytokine secretion profile of these Tr1 cell populations specific for type II collagen, cells were stimulated in the presence of anti-CD3+anti-CD28 monoclonal antibodies. ELISAs were performed on 48 h supernatants to measure IL-4, IL-10 and IFNγ production. FIG. 2 shows that the cytokine secretion profile observed for the latter type II collagen specific populations corresponds to a Tr1 cytokine secretion profile, i.e. high production of IL-10, low production of IFNγ and no production of IL-4.

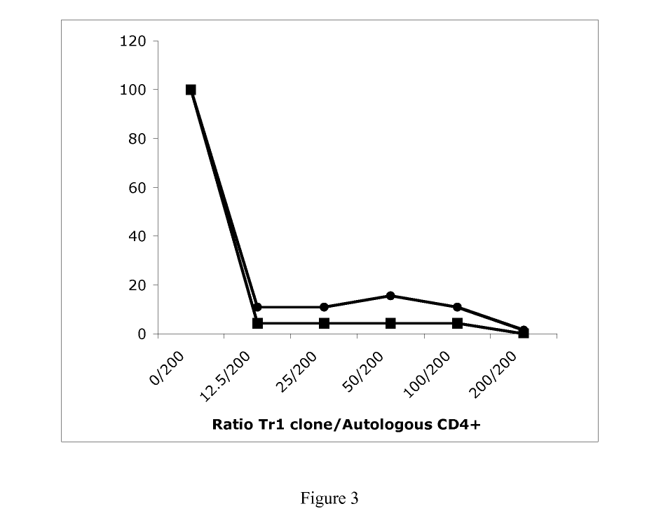

Suppressive activity of these type II collagen Tr1 populations was then assessed. Tr1 cells were co-cultivated with autologous CD4+ T cells in the presence of anti-CD3+anti-CD28 monoclonal antibodies. After 3 days of stimulation, cell proliferation was measured. FIG. 3 shows the results for the two Tr1 populations and confirms the suppressive activity of these cells.

### Example 2

### Human Versus Mouse Tr1 Cells Comparability

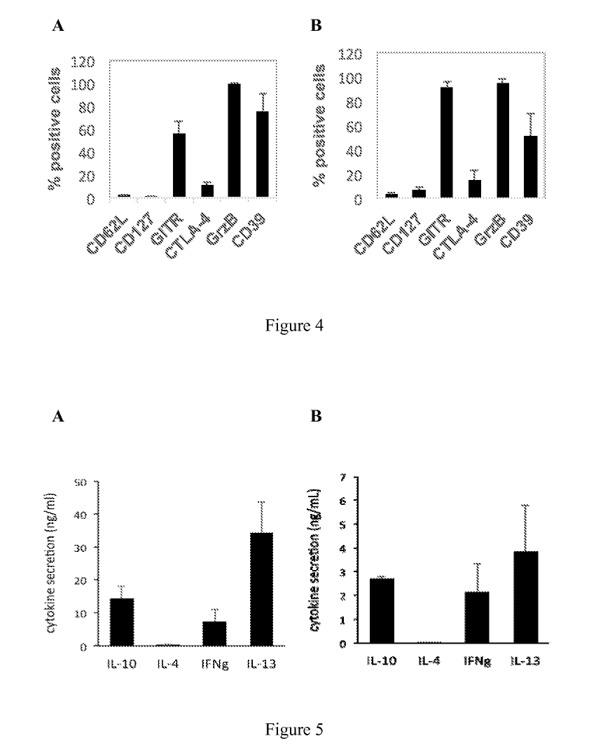

The mode of action of Tr1 cells directed to type II collagen implies recognition of collagen-II epitopes presented by antigen-presenting cells in the context of MHC class II molecules. This recognition triggers the immunomodulatory properties of the Treg cells of the invention. As a consequence animal studies are requiring the use of species-specific Treg cells allowing a perfect MHC match comparable to the autologous human use. In this context, and to allow in vivo pharmacology studies, Tr1 cells directed to type II collagen were produced from mice under the DBA-1 strain. FIG. 4 and FIG. 5 show the comparability of surface marker expression and cytokine secretion between human and mouse Tr1 cells directed to type II collagen.

The above FIG. 4 shows that Tr1 cells directed to type II collagen from the two species are comparable in terms of surface expression of Treg markers (low expression of both CD62L and CD127 as hallmark of induced Treg cells) and in terms of suppressive molecules expression (low expression of CTLA-4 and high expression of GITR, CD39 and Granzyme B).

In addition, evaluation of the cytokine secretion of mouse and human Tr1 cells directed to type II collagen also shows comparability between species with presence of IL-10, IFNγ and IL-13 secretion and absence of IL-4 secretion (FIG. 5).

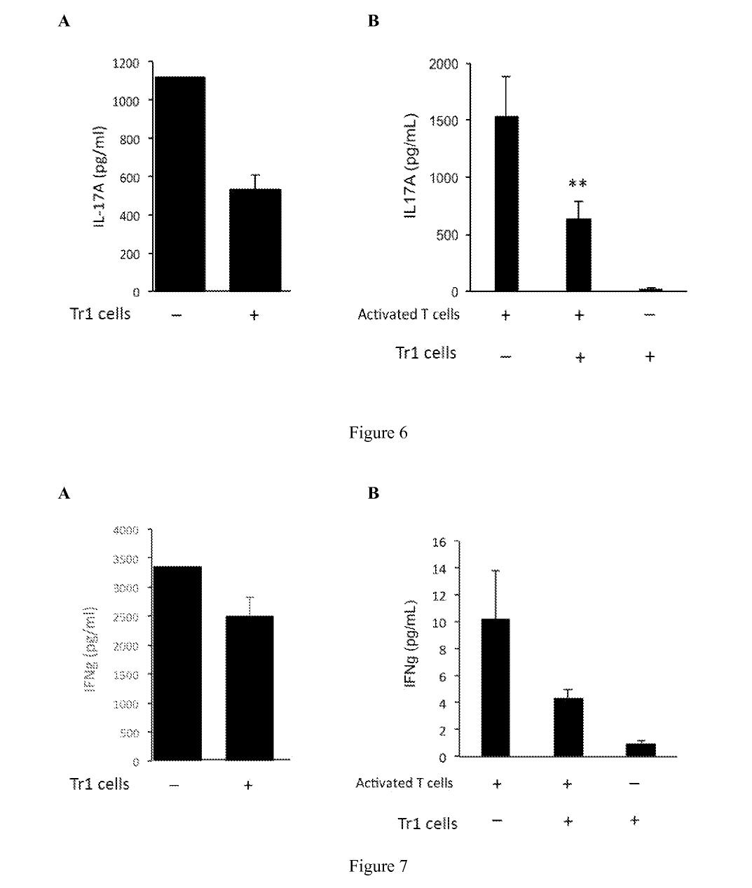

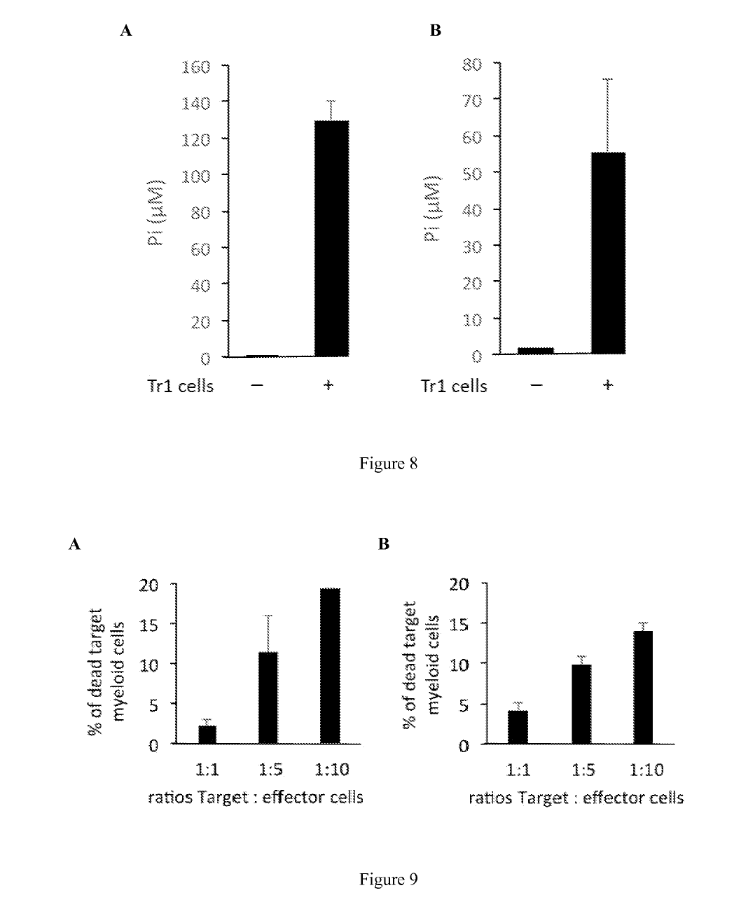

To demonstrate the immunomodulatory effect of Tr1 cells directed to type II collagen, in vitro suppression assays were performed with the aim of examining the different modes of Tr1 cells directed to type II collagen suppression. These tests were carried out for both human and mouse Tr1 cells directed to type II collagen. FIGS. 6 to 9 show that Tr1 cells directed to type II collagen are able to inhibit the IL-17 and IFNγ secretion by activated T cells (FIGS. 6 and 7), to hydrolyse extracellular ATP (FIG. 8) and to exert a cytotoxic effect on myeloid target cells (FIG. 9).

These results confirm the comparability of human and mouse Tr1 cells directed to type II collagen at the functional levels. In addition, it also confirms that Tr1 cells directed to type II collagen exert their suppressive effects using different modes of action and on multiple cellular and molecular targets through:
1- Soluble factor secretion allowing inhibition of pro-inflammatory cytokine secretion by activated T cells. Notably, Tr1 cells directed to type II collagen inhibit IL-17 secretion by effector cells. As IL-17 is known to be a pro-inflammatory effector molecule in human non-infectious uveitis, this activity of Tr1 cells directed to type II collagen supports the plausibility of Tr1 cells directed to type II collagen benefit to treat this disease. Also, both mouse and human Tr1 cells directed to type II collagen inhibit the secretion of IFNγ, a cytokine produced by pro-inflammatory uveitogenic Th1 type T cells,
2- ATP hydrolysis allowing reduction of extracellular ATP proinflammatory action and,
3- Myeloid cell killing allowing elimination of proinflammatory macrophages and dendritic cells known to perpetuate the inflammatory response in non-infectious uveitis.

### Example 3

### Treatment of Non-Infectious Uveitis

Model (EAU=Experimental Autoimmune Uveitis)

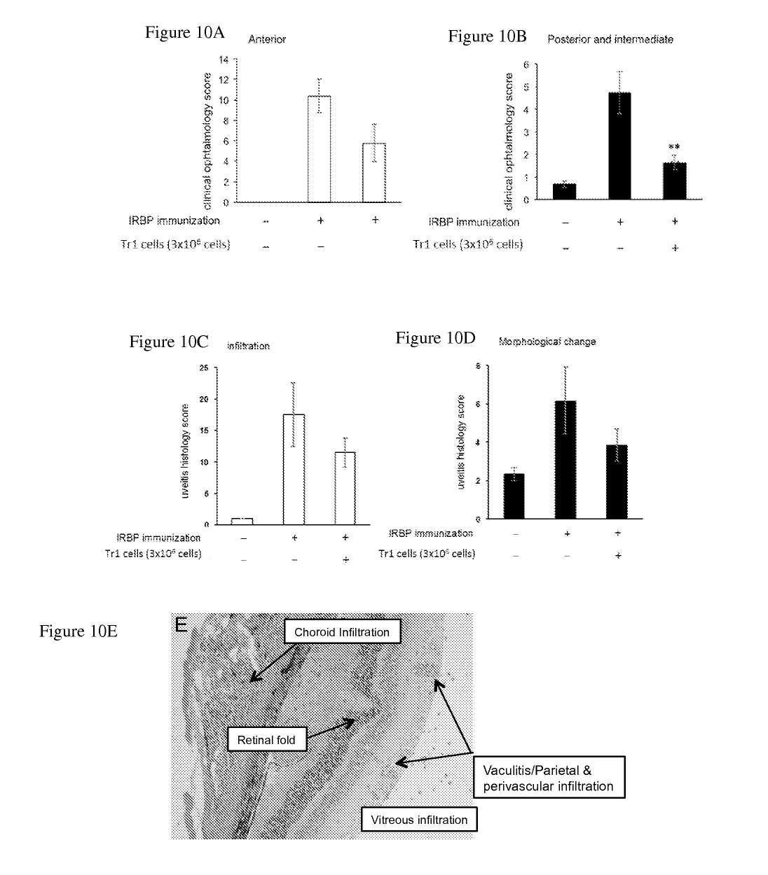

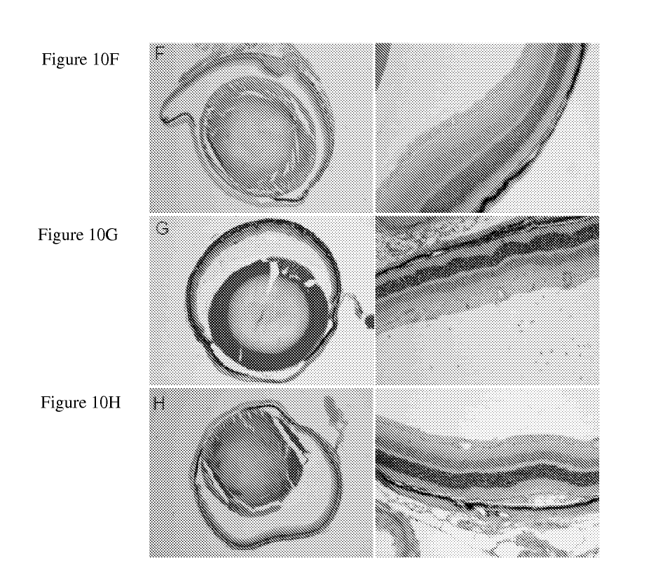

Experimental Autoimmune Uveitis (EAU) represents today the more common rodent model mimicking human non-infectious uveitis. Classically in EAU, the eye autoimmune inflammation is induced by immunization with retinal antigens in the presence of adjuvant. EAU was optimized in the DBA-1 strain that, although weakly sensitive to EAU induction, developed signs of anterior, intermediate and posterior inflammatory uveitis after immunization with human IRBP (Inter Photoreceptor Binding Protein) peptides in Complete Freund Adjuvant and Pertussis Toxin. 3.10⁶ Tr1 cells directed to type II collagen (produced on DBA-1 strain) were administered intravenously in the tail vein to EAU prone mice 8 days after the induction of uveitis. The inflammatory signs assessed by ophthalmoscopy and the histologic features of eyes taken from EAU mice treated or not with Tr1 cells directed to type II collagen were evaluated at Day 15 of the experiments (FIG. 10). Clinical and Histology scores are based on published literature (Copland et al, Investigative Ophthalmology and Visual Science, December 2008, 49(12): 5458-5465).

Results

FIG. 10 shows that DBA-1 mice immunized with IRBP peptides develop an acute uveitis characterized at Day 15 by an acute anterior uveitis (iris inflammation, inflammatory precipitates in the anterior chamber, dilatation of perilimbal vessels) together with a vitritis (vitreous haze due to inflammatory infiltrate) and a retinitis (vasodilatation and vasculitis, retinal folds). These clinical observations were confirmed at the histology level. Administration of Tr1 cells directed to type II collagen 8 days after the first immunization allows to significantly decrease posterior and intermediate clinical signs as well as anterior uveitis and allow decreasing eye infiltration as well as morphological changes of the retinal layer.

These in vivo results confirm, in the most relevant model of non-infectious uveitis in rodents, the potential of Tr1 cells directed to type II collagen to treat this pathology. Indeed, a beneficial effect of Tr1 cells directed to type II collagen was observed not only on clinical signs but also by histology, an objective measurement of the inflammatory score. This animal data are the most relevant example of a plausible benefit of Tr1 cells directed to type II collagen for the treatment of non-infectious uveitis.

### Example 4

### Manufacturing Process of Tr1 Cells Directed to Type II Collagen

Tr1 Cells Enrichment

150 ml of patient's peripheral whole blood (starting material) are collected in a sterile container by standard venipuncture. Then, Peripheral Blood Mononuclear Cells (PBMC) are isolated by Ficoll gradient density centrifugation and cultivated in the presence human type II collagen for several days.

Selection of Tr1 Cells Directed to Type II Collagen by Cloning

Activated PBMCs are harvested and diluted at a low concentration. T cells are stimulated with anti-CD3 monoclonal antibody (mAb) and anti-CD28 mAb. After approximately 3 weeks, wells showing growth-positive T cells are macroscopically identified, followed by microscopic examinations and cells suspensions are further expanded.

Expansion

Before starting the expansion step, only the cells suspensions demonstrating specificity to human collagen-II by showing a release of IL-10 specifically in response to collagen-II are selected for processing. CD4⁺ collagen-II-specific cells suspensions are then expanded. During this expansion step, other in-process controls are performed in order to ensure the type 1 Treg profile of the cells suspensions by evaluating the cytokine secretion profile (IL-10, IFNγ, IL-4 and IL-13) after polyclonal activation (anti-CD3+anti-CD28mAb coated beads).

### Example 5

### Detection of Collagen-II Specific Tr1 Cells by Quantitative PCR

For the detection of Collagen-II specific Tr1 cells by PCR, primers and Dual label FAM-TRAM probe (SIGMA) were designed for the specific TCR V-D-J sequence expressed by collagen-II specific Tr1 cells. Quantitative PCRs were performed on total tissue derived genomic DNA extracted using GenElute mammalian gDNA kit (SIGMA). Data from real-time quantitative PCR using detection system (Pikoreal, Thermo) were expressed as the number of collagen-II specific Tr1 cells determined based on standard curves using dilution of Collagen-II specific Tr1 cells diluted in non-transgenic cells expressed by mg of tissues or per organ (for small samples ie eye).

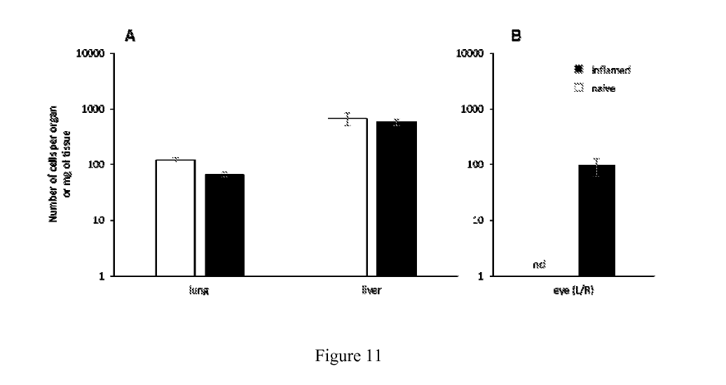

FIG. 11 shows that, 24-hours after intravenous injection, collagen-II specific Tr1 cells injected intravenously are detected in the lung and liver in mice with uveitis (inflamed mice) and without uveitis (naïve mice) uveitis (Panel A). In contrast, collagen-II specific Tr1 cells are only detected in the eye of inflamed mice (Panel B). These results demonstrate the Tr1 cells systemically administered migrates to the site of inflammation.

1. A method for treating uveitis in a subject in need thereof, comprising administering an effective amount of human regulatory T cells (Treg cells) directed to an eye-associated antigen to the subject.

2. The method according to claim 1, wherein said eye-associated antigen is selected from the group comprising type II collagen, retinal arrestin, S-arrestin, interphotoreceptor retinoid-binding proteins (IRBP1), betaB1-crystallin, retinal proteins, and choroid proteins, and fragments, variants and mixtures thereof.

3. The method according to claim 1, wherein said eye-associated antigen is type II collagen and fragments, variants and mixtures thereof.

4. The method according to claim 1, wherein uveitis is non-infectious uveitis.

5. The method according to claim 1, wherein uveitis is chronic uveitis.

6. The method according to claim 1, wherein uveitis is recurrent uveitis.

7. The method according to claim 1, wherein uveitis is panuveitis, or intermediate and/or posterior uveitis.

8. The method according to claim 1, for treating macular oedema related to uveitis.

9. The method according to claim 1, wherein human Treg cells are autologous to the cells of said subject.

10. The method according to claim 1, wherein 10⁴ to 10⁷ Treg cells are administered to the subject in need thereof.

11. The method according to claim 1, wherein the administration to said subject of an effective amount of Treg cells directed to an eye-associated antigen is in combination with one or more therapeutic agents used for treating uveitis.

12. The method according to claim 1, wherein the administration to said subject of an effective amount of Treg cells directed to an eye-associated antigen is in combination with one or more therapeutic agents selected in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

13. The method according to claim 1, wherein the administration to said subject of an effective amount of Treg cells directed to an eye-associated antigen is in combination with human MSC.

14. The method according to claim 1, wherein said subject does not respond adequately to, or is unlikely to respond adequately to, one or more therapeutic agents in the group of a steroid or a corticosteroid (such as, for example, betamethasone, cortisone, hydrocortisone, deflazacort, dexamethasone, fludrocortisone, fluocinolone, fluorometholone, methylprednisolone, prednisone, prednisolone, rimexolone, triamcinolone, rimexolone, difluprednate), CF-101 (an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, Sarilumab (anti-IL-6R monoclonal antibody), Secukinumab (anti-IL-17A monoclonal antibody), Sirolimus (mTOR inhibitor), Voclosporin, Gevokizumab (IL-1 beta antagonist), Humira (adalimumab—anti-TNF monoclonal antibody), Homatropine (muscarinic receptor antagonist), Tsiklopentolat (muscarinic receptor antagonist), Atropine sulfate (muscarinic receptor antagonist), methotrexate, azathioprine, acyclovir, gentamycin, neomycin, polymyxin B, rolitetracycline, sulfacetamide, valacyclovir, chloramphenicol, mycophenolate, a combination of fluocinolone and neomycin, a combination of neomycin, polymyxin B and prednisolone, a combination of prednisolone and sulfacetamide, or mixtures thereof.

15. Method for treating uveitis in a subject in need thereof, said method comprising the steps of:
obtaining Treg cells directed to a selected eye-associated antigen, said Treg cells being obtained from a blood sample of said subject,
further expanding Treg cells obtained at the previous step,
injecting Treg cells thus obtained in said subject, preferably by intravenous route.
